(19) United States
(12) Patent Application Publication
(10) Pub. No.: US 2009/0053106 A1
(43) Pub. Date: Feb. 26, 2009

(54) Autonomous microfluidic apparatus

(75) Inventors: Jhy-Wen Wu, Hsinchu City (TW); Hung-Jen Yang, Hsinchu City (TW); Nan-Kuang Yao, Taoyuan County (TW)

Correspondence Address: BIRCH STEWART KOLASCH & BIRCH, PO BOX 747, FALLS CHURCH, VA, 22040-0747, US

(73) Assignee: Industrial Technology Research Institute

(21) Appl. No.: 12/007,931

(22) Filed: Jan. 17, 2008

(30) Foreign Application Priority Data
Aug. 23, 2007 (TW) 096131165

(51) Int. Cl. G01N 33/00

(52) U.S. Cl. 422/68.1

(57) ABSTRACT

The present invention relates to an autonomous microfluidic apparatus. The autonomous microfluidic apparatus is substantially a substrate having a microchannel structure arranged thereon. As a microfluid is being filled in a loading well situated upstream of the microchannel structure, the microfluid is affected by interactions between gravity, adhesive force and surface tension and thus driven to flow downstream in the microchannel structure while filling a plurality of manifolds formed in a area situated downstream of the microchannel structure, so that accurate and autonomous quantification and separation of the microfluid using the plural manifolds, each having a specific length, can be achieved and provided for biomedical inspection and analysis.

### FIELD OF THE INVENTION

The present invention relates to an autonomous microfluidic apparatus, and more particularly, to an inexpensive and easy-to-manufactured apparatus capable of separating a microfluid in an autonomous manner by subjecting the microfluid under interactions between gravity, adhesive force and surface tension for driving the same to flow in a microchannel structure formed in the apparatus, and thus to be adapted for related microfluidic industry, such as biomedical inspection and biochemical analysis.

### BACKGROUND OF THE INVENTION

Nowadays, it is more and more common to use microfluidic devices in biochemical analysis that microfluidic devices form the base for a range of biotechnical and chemical applications with a huge market potential. Depending on the application, it may lead to less reagent and power consumption, increased performance and faster analysis with higher precision, higher sample throughput, easier integration and automation with less manpower consumption. However, because of the direct consequence of miniaturization, microfluidic devices are used to deal with matters in a world with a physical scale between a couple of millimeters and the submicron scale, which can be referred as the microworld. The microworld differ from the macroworld that we perceive in daily life in the scale of a couple of kilometers down to a part of a millimeter, since they are dominated by difference forces. Therefore, from an engineering point of view, it is important to control the flowing of microfluid in microfluidic devices in every situation where using the benefits of the physical scaling laws of the microworld in terms of performance or cost.

For most biochemical analyses, the microfluidic devices should be designed with the following basic capabilities:
(1)they should be able to process the flowing of at least three to five microfluids;
(2)they should be able to regulate the flowing of the at least three to five microfluids according to a specific order;
(3)they should be able to defined the amount of the at least three to five microfluids being filled into the microfluidic devices;
(4)while filling two microfluids into the microfluidic devices one after another according to the specific order, they should be able to prevent the two successive microfluids from mixing with each other.
   However, the aforesaid capabilities are only basic requirements regarding to the designing of microfluidic devices, it is preferred to control and perform a number of chemical processes on a single microfluidic chip in batch processing.

In order to control and perform a number of chemical processes on a single microfluidic chip in batch processing, it is required to split and separate a flow into a plurality of sub-flows while maintaining the stability of each sub-flow without mixing with each other. Not to mention that it should be able to prevent two microfluids from mixing with each other while filling the two microfluids into the microfluidic devices one after another according to the specific order. Currently, a conventional microfluidic chip is an integrated device composed of various micro electromechanical system (MEMS) components, such as micro pumps, micro valves, microchannel layouts, flow sensors, micro flow switches and differential pressure actuators. If any one of such MEMS components malfunction or is defected, the integrated microfluidic chip will not be able to function adequately, not to mention it is difficult to fabricate those various MEMS components on a single chip. Moreover, such conventional microfluidic chips require to be connected to various external electromechanical devices for supporting the same to operate properly, so that they can not function as personalized, disposable biomedical microfluidic chips with bedside testing ability.

Please refer to FIG. 1, which shows a microfluidic chip disclosed in TW Pt. No. 90130420, entitled “Chip for counting, classifying and analyzing microfluids and the manufacturing method thereof”. The aforesaid microfluidic chip is configured with three sample flow microchannels 171, 172, 173, four sheath flow microchannels 18, and none exiting microchannels 19, by which as sample flows of microfluidic are being filled into the sample flow microchannels 171, 172, 173 by the driving of a computer-controlled pump, the sample flows as well as the flows inside the sheath flow microchannels 19 are converged to a specific width, such as the width of a cell, for facilitating the same to be detected by the optical beams a, b, c, d.

Please refer to FIG. 2, which shows a microfluidic chip disclosed in TW Pt. No. 91121297, entitled “Network-type Microfluidic Apparatus”. The microfluidic apparatus 21 is composed of two main channel 211, 212 and three sub-channels 213, 214, 215, in which of the widths of the two main channels 211, 212 are defined as W1 and W2 in respective, and the widths of the three sub-channels 213, 214, 215 are defined as W3, while defined W1=W3>W2. As soon as an enzyme 46 is dripped into a loading well 216 of the aforesaid microfluidic apparatus 21, it is driven to flow into the main microchannel 211 by the interaction of surface tension relating to the channel width design, and then flow into the sub-channels 213, 214, 215. Moreover, the inner wall of each microchannel is hydrophile processed by a plasma surface process so as to ensure the enzyme 46 to combine well with the microchannel. Furthermore, since the microfluidic apparatus 21 is levelly disposed, the main microchannels 211, 212, and the sub-channels 213, 214, 215 are positioned at the same altitude and thus are different only in their width, it is required to designed an acquisition distribution layer at the inlet of each sub-channel 213, 214, 215 for so as to ensure the enzyme 46 to flow smoothly into those sub-channels 213, 214, 215.

Please refer to FIG. 3, which is a microfluidic chip being formed by electroplating and stamping microchannels on an optical disc, disclosed in “Design Fabrication of Polymer Microfluidic Platforms for Biomedical Application,” ANTEC-SPE 59^{th}, vol. 3, 2001. by M. J. Modau et al. In FIG. 3, as there are a plurality of capillary valves 2 formed on a rotary table 1, microfluid filled in microchannels of different radiuses can be selected to flowing into a reaction chamber by changing the rotation speed of the rotary table 1. However, the aforesaid device is disadvantageous in that: it is required to have those capillary valves 2 which can cost additional cost and design difficulty, and the rotary table 1 rotating in high speed might cause undesirable vibration.

Furthermore, there is another current available microfluidic chip, disclosed in a paper named “Optical Microfluid Control Based on Potoresponsive Polymer Gel Microvalves” by Shinji Sugoura et al. which is designed to have its microfluid valve to be formed by a photoresponsive polymer. In which, as the microfluid valve can response to the shining of light and thus open, the flowing of microfluid can be controlled. However, it is disadvantageous in that: each microfluid valve can be controlled to open only once.

Therefore, it is required to have a low-cost, simple-structured microfluidic apparatus capable of automatically and accurately separating samples by a simple process without the driving of a power source, movable valves and the support of external electromechanical devices.

### SUMMARY OF THE INVENTION

Embodiments of the present invention provide an inexpensive and easy-to-manufactured autonomous microfluidic apparatus, capable of separating a microfluid in an autonomous manner by subjecting the microfluid under interactions between gravity, adhesive force and surface tension for driving the same to flow in a microchannel structure formed in the apparatus, which can be adapted for various microfluidic system in applications, such as biomedical inspection and biochemical analysis, etc.

One of the present invention provides an autonomous microfluidic apparatus, comprising:
a substrate; and
a microchannel structure, arranged on the substrate and further comprising:
a main microchannel;
a loading well, formed on the main microchannel;
a plurality of manifolds, each channeling with the main microchannel;
at least a passive valve, each being disposed at the main microchannel at a position between any two neighboring manifolds of the plural manifolds; and
a plurality of restriction areas, formed at ends of the plural manifolds.

In an exemplary embodiment of the invention, the depth of the main microchannel is different from those of the plural manifolds.

In another exemplary embodiment of the invention, the lengths of the plural manifolds are not the same.

In another exemplary embodiment of the invention, the plural manifolds are arranged parallel with each other.

In another exemplary embodiment of the invention, the loading well is connected to at least a via hole, provided for exerting a specific pressure to the microfluid in the loading well.

In another exemplary embodiment of the invention, the cross section area of each restriction area is different from that of the manifold where it is connected with.

In another exemplary embodiment of the invention, the passive valve can be a recess.

In another exemplary embodiment of the invention, the main microchannel is configured with a waste well, being an area situated at a downstream end of the main microchannel and filled with a material selected from the group consisting of a polymer fiber, materials with water absorption ability, and the combination thereof.

In another exemplary embodiment of the invention, the cross section area of the waste well is different from that of the main microchannel where it is connected with.

In another exemplary embodiment of the invention, an exiting microchannel is arranged at a position between the main microchannel and the waste well in a manner that it is extending perpendicular to the main microchannel.

In another exemplary embodiment of the invention, the exiting microchannel is extending parallel to the plural manifolds.

In another exemplary embodiment of the invention, a passive valve is arranged at the exiting microchannel at a position proximate to the waste well.

In another exemplary embodiment of the invention, the cross section area of the passive valve is different from those of the exiting microchannel and the waste well where it is connected with.

In another exemplary embodiment of the invention, the passive vale connected to the exiting microchannel can be a recess.

In another exemplary embodiment of the invention, the main microchannel is filled with a material selected from the group consisting of a polymer fiber, materials with water absorption ability, and the combination thereof.

In another exemplary embodiment of the invention, the substrate is a flat plate having the main microchannel to be formed thereon in equal depth.

In another exemplary embodiment of the invention, the microfluidic apparatus further comprises: a slope structure, used for sloping the substrate and thus forming an included angle between the sloped substrate and a datum water level so as to slope the main microchannel from the downstream side thereof to the upstream side thereof with increasing height according to the included angle.

In another exemplary embodiment of the invention, each of the plural manifolds is extending about perpendicular to the main microchannel.

Further scope of applicability of the present application will become more apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The present invention will become more fully understood from the detailed description given herein below and the accompanying drawings which are given by way of illustration only, and thus are not limitative of the present invention and wherein:

FIG. 1 shows a microfluidic chip disclosed in TW Pt. No. 90130420, entitled “Chip for counting, classifying and analyzing microfluids and the manufacturing method thereof”.

FIG. 2 shows a microfluidic chip disclosed in TW Pt. No. 91121297, entitled “Network-type Microfluidic Apparatus”.

FIG. 3 shows a conventional microfluidic chip, being formed by electroplating and stamping microchannels on an optical disc.

FIG. 4 is a three-dimensional diagram showing a microfluid flowing in a microchannel.

FIG. 5 is a front view of an autonomous microfluidic apparatus according to a first embodiment of the invention.

FIG. 5A is an A-A cross sectional view of FIG. 5.

FIG. 5B is a B-B cross sectional view of FIG. 5.

FIG. 6 shows an autonomous microfluidic apparatus of the invention, being slope-disposed with respect to a datum water level.

FIG. 7(a)˜(d) shows a microfluid being separated in an autonomous microfluidic apparatus of the invention.

FIG. 8 is a front view of an autonomous microfluidic apparatus according to a second embodiment of the invention.

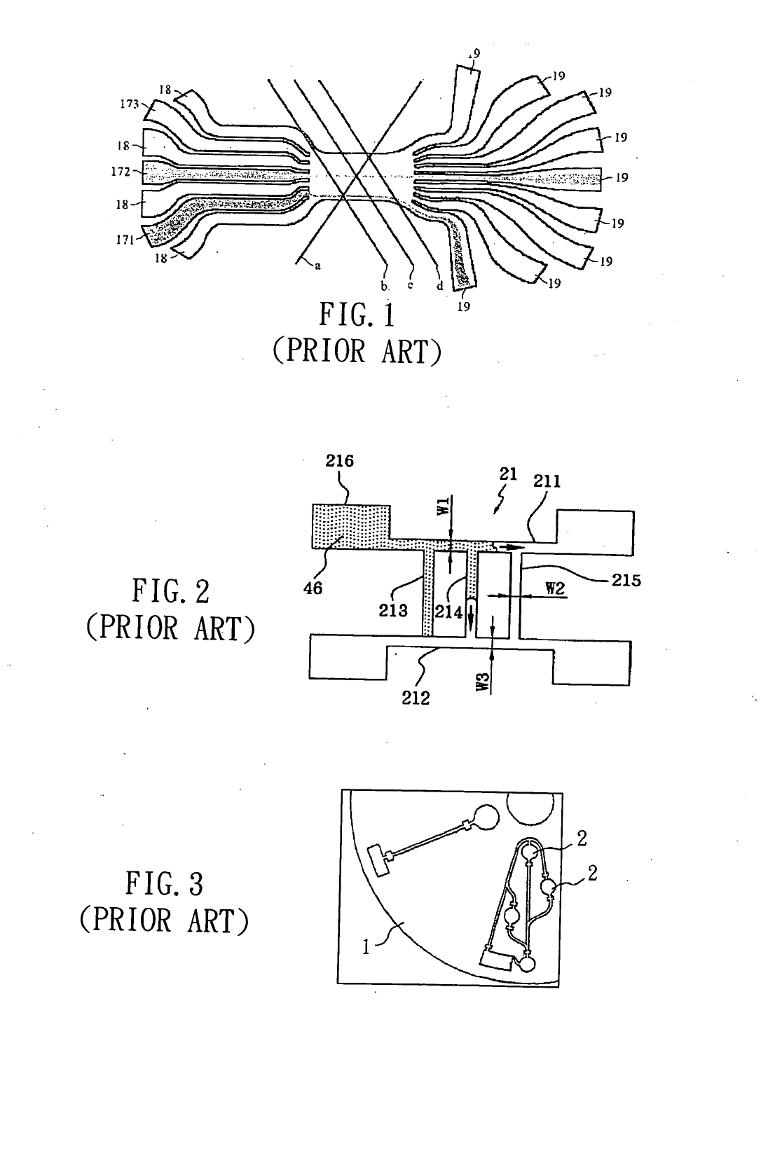

### DESCRIPTION OF THE EXEMPLARY EMBODIMENTS

For your esteemed members of reviewing committee to further understand and recognize the fulfilled functions and structural characteristics of the invention, several exemplary embodiments cooperating with detailed description are presented as the follows.

It is intended to design an autonomous microfluidic apparatus capable of automatically and accurately separating samples while driving the separated sample by gravity to flow in a microchannel structure and into reaction areas in respective. However, as such microfluidic apparatus is working in the so-called microworld, one direct consequences of miniaturization is that the surface to volume ratio increases linear with decreasing feature size, i.e. the relatively large surfaces in the microworld result in increased physical interaction between the different material phases which gives some interesting challenges and a range of possibilities. In detail, when a microfluid is driven by gravity to flow in a main microchannel of the microfluidic apparatus, the flowing microfluid is greatly influenced by surface tension due to the change of interface free energy between liquid phase-gas phase-solid phase, and thus, by changing the microchannel structure or the surface texture of the microchannel, passive valves can be formed and used for altering the flowing direction of the microfluid while directing the microfluid to flow into a plurality of manifolds in respective, i.e. the reaction areas. Thereafter, as soon as each reaction areas is filled with the microfluid and all the reactions required to be performed are complete, the microfluid is driving to flow out of the reaction areas by the absorbing force of a waste area. In addition, as the main microchannel of the microfluidic apparatus is filled with a material with water absorption ability, such as a hydrophile polymer fiber, which is capable of generating a pulling force to resist the gravity, and no such material is used to filled the manifolds, microfluid filled in the manifold will be pulled by the gravity to flow toward the waste area faster than the main microchannel. Therefore, the aforesaid autonomous microfluidic apparatus is able to separate microfluid automatically and accurately. The basic design principle of the autonomous microfluidic apparatus is described hereinafter.

When a microfluid is flowing in a microchannel, its total free surface energy can be represented as:

U_{T}=A_{SL}γ_{SL}+A_{SG}γ_{SG}+A_{LG}γ_{LG} (1)

wherein A_{SL} represents the area of solid-liquid interface;
A_{SG} represents the area of solid-gas interface;
A_{LG} represents the area of liquid-gas interface;
γ_{SL} represents the surface tension per unit length at solid-liquid interface;
γ_{SG} represents the surface tension per unit length at solid-gas interface;
γ_{LG} represents the surface tension per unit length at liquid-gas interface.
   When a drop of liquid drips on a solid surface, an angle θ_{c} will be formed on the liquid-solid interface, which is referred as the contact angle at liquid-solid interface. Accordingly, Young's equation can be used for describing the relationship between solid-liquid, solid-gas, and liquid-gas interface energies, as following:

γ_{SG}=γ_{SL}+γ_{LG}cos θ_{c} (2)

By substituting equation (2) into equation (1) and partial differentiating the total free surface energy U_{T} by wet volume V_{L}, capillary pressure P on the liquid can be obtain as:

$$P = - \frac{d {U}_{T}}{d {V}_{L}} = {γ}_{L G} ( cos {θ}_{c} \frac{d {A}_{S L}}{d {V}_{L}} - \frac{d {A}_{L G}}{d {V}_{L}} ) \qquad ( 3 )$$

From equation (3), the pressure p for driving the liquid to move is related to the variation between the total surface free energy and the wet volume. Therefore, a passive valve can be generated either by controlling the total surface free energy or by controlling the wet volume according to equation (3).

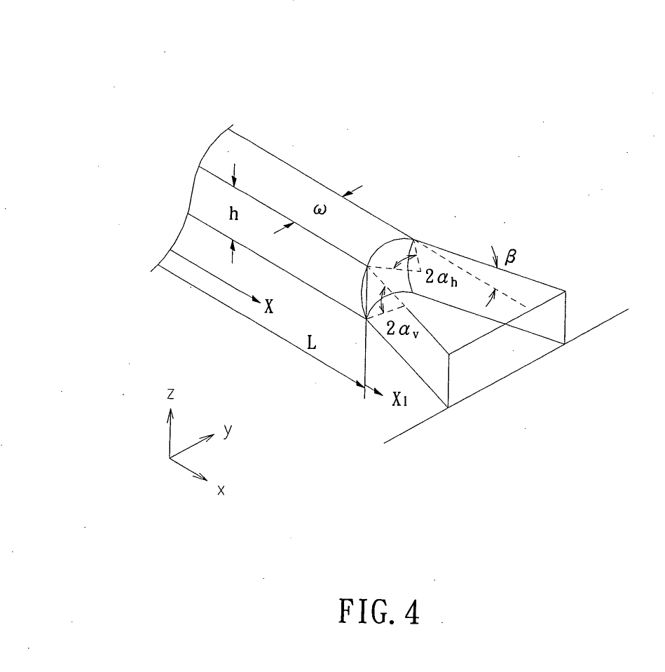

The foregoing description only relates to two-dimensional model. For describing a microfluid flowing in a microchannel in actual three-dimensional model, it is assumed that the front of the flow can be represented as two perpendicular crescents, as shown in FIG. 4, so that the total surface free energy can be represented as:

$${U}_{T} = {U}_{0} - {γ}_{L α} cos {θ}_{c} [ \begin{matrix}2 L ( w + h ) - \frac{{w}^{2}}{2 sin {α}_{h}} \\ ( \frac{{α}_{h}}{sin {α}_{h}} - cos {α}_{h} )\end{matrix} ] + {γ}_{L α} \frac{w h {α}_{h} {α}_{v}}{sin {α}_{h} sin {α}_{v}} \qquad ( 4 )$$

wherein, the wet volume is as following:

$${V}_{L} = w l h - \frac{{w}^{2} h}{4 sin {α}_{h}} ( \frac{{α}_{h}}{sin {α}_{h}} h - cos {α}_{h} ) - \frac{w {h}^{2} {α}_{h}}{4 sin {α}_{v} sin {α}_{h}} ( \frac{{α}_{v}}{sin {α}_{v}} - cos {α}_{v} ) \qquad ( 5 )$$

From the aforesaid equation (4) and equation (5), it can be concluded that the design of passive valves in microchannel are most significantly related to the following three parameters:
(1) the depth h of the microchannel;
(2) the width w of the microchannel; and
(3) the extending angle β relating to the extending of the microchannel.
   Accordingly, an autonomous microfluidic apparatus capable of separating a microfluid automatically and accurately can be achieved by incorporating the microchannel design of the aforesaid parameters and the interaction between gravity and absorption of the fillers in its microchannel.

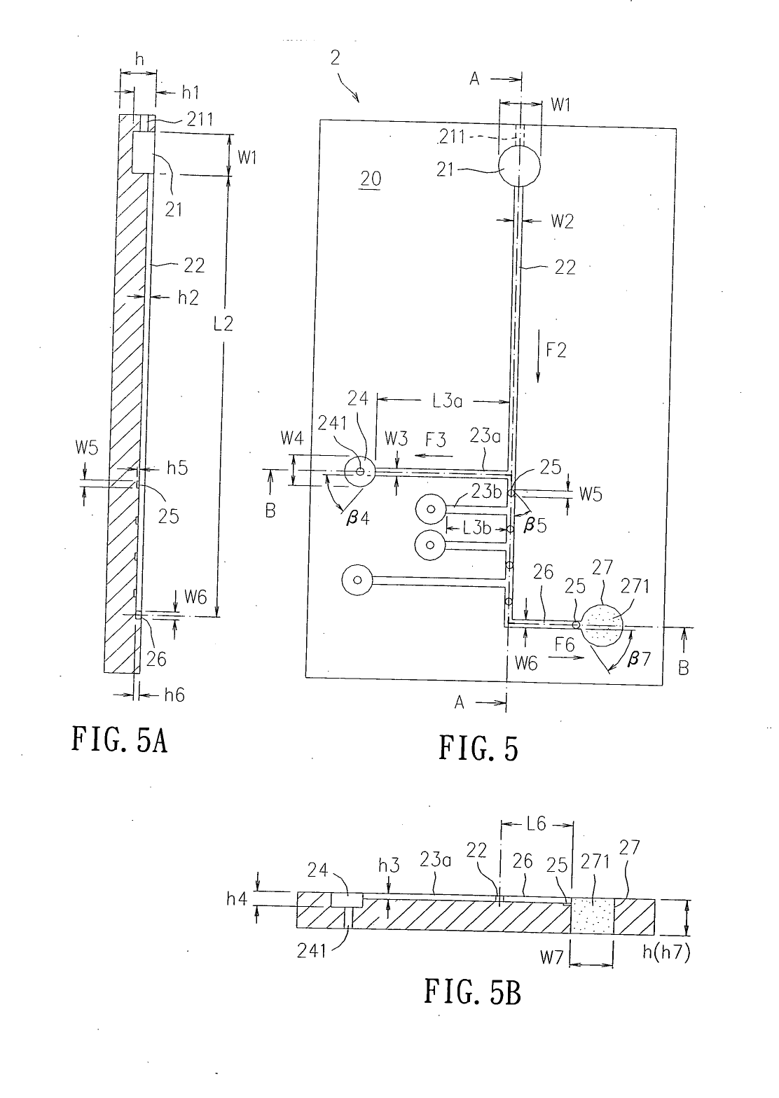

Please refer to FIG. 5, FIG. 5A and FIG. 5B, which show an autonomous microfluidic apparatus according to a first embodiment of the invention. The autonomous microfluidic apparatus 2 is substantially a substrate 20 having a microchannel structure arranged thereon. The microchannel structure comprises: a main microchannel 22; a plurality of manifolds 23a, 23b, parallel-arranged beside the main microchannel 22; wherein, the main microchannel is filled with a material selected from the group consisting of a polymer fiber, materials with water absorption ability, and the combination thereof. In addition, the substrate 20 can be made of a plastic with certain rigidity, such as polymethylmethacrylate (PMMA); and the microchannel structure is formed on the substrate 20 by milling and the cross section area of the microchannel structure is ranged between 0.1 micrometer and 1000 micrometer, which is dependent upon the microfluid to be applied.

The main microchannel 22 is extending parallel with a longitudinal axial direction F2 of the substrate 20 and is substantially a groove of L2 length, W2 width and h2 depth. There is a loading well 21 formed at the top of the main microchannel 22 which is a circular concave of W1 diameter and h1 depth. The loading well 21 is designed for receiving a specific amount of microfluid sufficient enough to flow into the main microchannel 22 for separation, so that the diameter W1 and depth h1 of the loading well 21 are all larger than the width W2 and depth h2 of the main microchannel 22. Moreover, for facilitating the microfluid to flow into the main microchannel 22 from the loading well 21 smoothly, a via hole 211 is formed on the substrate 20 in a manner that it channels the loading well with its ambient environment so as to enable the microfluid received in the loading well 21 to be subjected to the atmospheric pressure and thus exerting a specific pressure to the microfluid for pressing the same to flow out of the loading well 21 smoothly.

In addition, there is an exiting microchannel 26 arranged at the tail of the main microchannel 22, i.e. at the end of the main microchannel 22 far from the loading well 21. The exiting microchannel 26 is extending following a direction F2 perpendicular to the longitudinal axial direction F2 of the substrate 20 and is substantially a groove of L6 length, W6 width and h6 depth, in which the length L6 may be different from the length L2 of the main microchannel 22, but the width W6 and depth h6 are the same as the width W2 and depth h2 of the main microchannel 22. As an end of the exiting microchannel 26 is connected to the main microchannel 22, the other end of the exiting microchannel 26 is configured to connect to a waste well 27 which is substantially a circular concave of W7 diameter and h7 depth. The waste well 27 is so-designed for enabling its diameter W7 and depth h7 to be larger than the width W6 and depth h6 of the exiting microchannel 27 while forming an extending angle β7 relating to the circular-shaped waste well 27 and the width W6 of the exiting microchannel 26. As shown in FIG. 5B, the depth h7 of-the waste well 27 is equal to the thickness h of the substrate 20 so that the waste well 27 can be a hole on the substrate 20. It is noted that the waste well 27 can be stuffed with a material selected from the group consisting of a polymer fiber, materials with water absorption ability, and the combination thereof.

The plural manifolds 23a, 23b are parallel-arranged beside the main microchannel 22 which are extending following a direction F3 perpendicular to the main microchannel 22. In this embodiment, the manifold 23a substantially a groove of L3a length, W3 width and h3 depth, in which the width W3 is the same as the width W2 of the main microchannel 22 while its depth h3 may or may not be the same as the depth h2 the main microchannel 22. The only difference between the manifold 23b and the manifold 23a is that: the length L3b of the manifold 23b is shorter than that of the manifold 23a, so that the following description only use the manifold 23a as illustration. As an end of the manifold 23a is connected to the main microchannel 22, the other end of the manifold 23a is configured to connect to a restriction area 24 which is substantially a circular concave of W4 diameter and h4 depth. The restriction area 24 is so-designed for enabling its diameter W4 and depth h4 to be larger than the width W3 and depth h3 of the manifold 23a while forming an extending angle β4 relating to the circular-shaped restriction area 24 and the width W3 of the manifold 23a. In addition, a via hole 241 is formed inside the restriction area 24 which bores through the substrate 20 as shown in FIG. 5B. By the atmosphere pressure provided through the via hole 24 and the designing of the cross section area difference between the restriction area 24 and the manifolds 23a and 23b, the interactions between gravity, adhesive force and surface tension exerting on the microfluid flowing inside the manifolds 23a, 23b will cause the microfluid to flow in and out of the manifolds 23a and 23b smoothly. It is noted that the restriction area can be substantially a via hole. Moreover, an array of recesses 25 are formed on the main microchannel 22 from the upstream thereof to the downstream thereof following the extending direction F2, that each of which is disposed at the main microchannel 22 at a position between any two neighboring manifolds 23a, 23b. In addition, there is a recess 25 formed at the intersection of the exiting microchannel 26 and the waste well 27, which is a circular concave of W5 diameter and h5 depth. By the disposition of the recesses 25, the depth of the main microchannel 22 is undulated, as shown in FIG. 5A. Moreover, an extending angle β5 is formed relating to the circular-shaped recess 25 and the width W2 of the main microchannel 22.

The microchannel structure of the aforesaid microfluidic apparatus 2 is designed according to the three parameters, that is, the depth h, the width h and the extending angle β. However, in order to subject the microfluid flowing in such microchannel structure to gravity, the microfluidic apparatus 2 must be inclined.

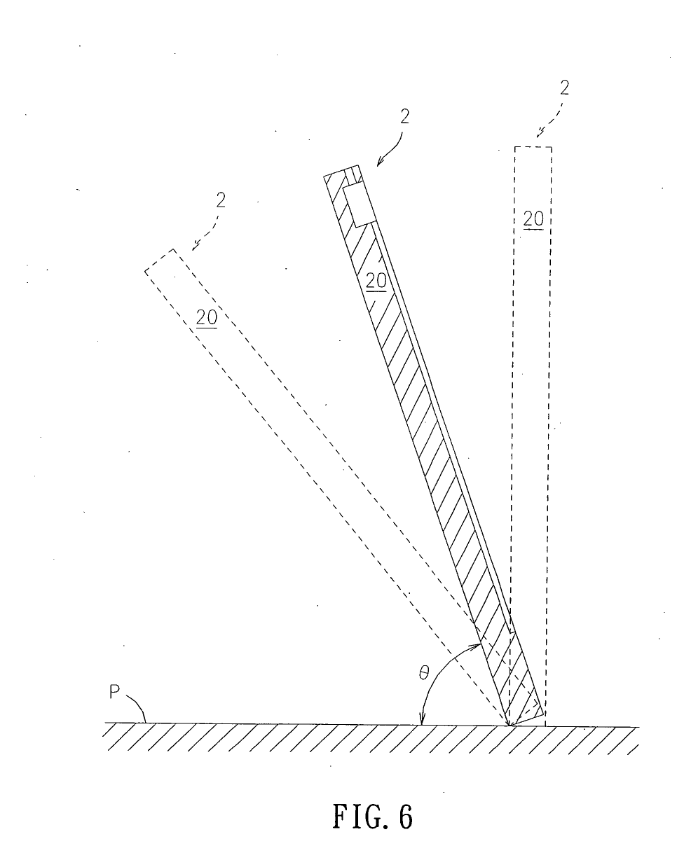

Please refer to FIG. 6, which shows an autonomous microfluidic apparatus of the invention, being slope-disposed with respect to a datum water level. As shown in FIG. 6, for enabling the autonomous microfluidic apparatus to slope by an angle θ with respect to a datum water level P, the upstream portion of the substrate 20 can raised by the use of an external structure or device (not shown in the figure) for forming an included angle θ between the substrate 20 and the datum water level P. Thereby, the microchannel structure formed on the substrate 20 is inclined and thus the microfluid can be force by gravity to flow from the upstream to the downstream of the microchannel structure. It is noted that the external structure or device used for tilting the substrate 20 can be a support platform or a support arm. Moreover, the sloping of the microchannel structure can be achieved by designing the surface of the substrate 20 to be a sloped surface, or by increasing the depth of the main microchannel from it upstream to downstream, but is not limited thereby. As the art for achieving the sloping is known to those skilled in the art, it is not described further herein. In this embodiment, as the substrate 20 is a flat plate, the sloping can be achieved by disposing an adjustable platform/strut at the bottom of the substrate, so that the included angle θ between the substrate 20 and the datum water level P can be adjusted by the adjustable platform/strut. The included angle θ can be ranged between 0 degree and 90 degrees if the microchannel structure is an open system formed on the surface of the substrate 20 so that the microfluid can be prevented from spilling out of the microchannel. However, if the microchannel structure is a closed system sealed inside the substrate 20, the included angle θ can be ranged between 90 degrees and 180 degrees.

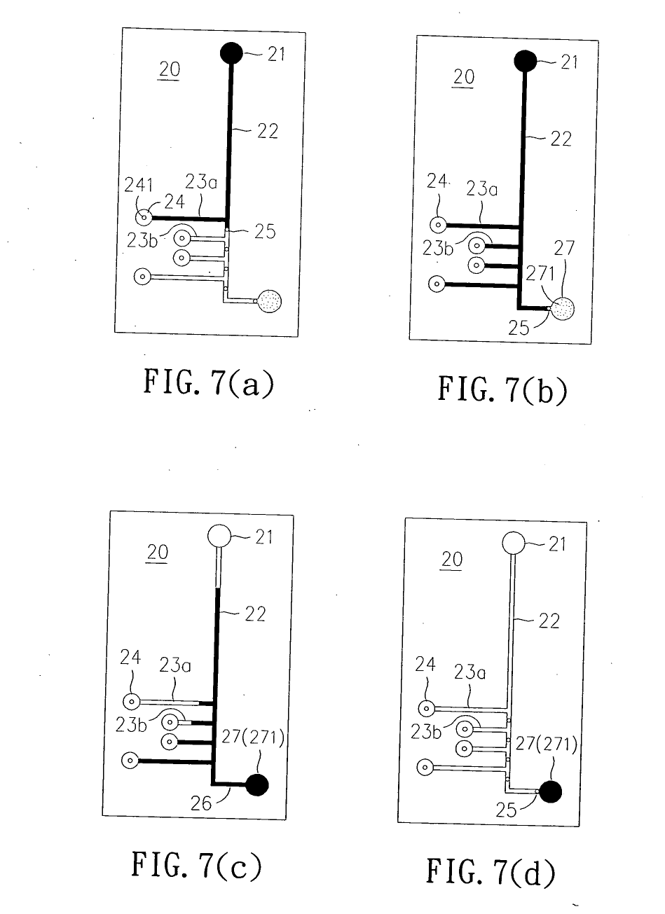

From the above description, it is known that the microfluid in the aforesaid microfluidic apparatus 2 is flowing successively from the loading well 21, the main microchannel 22, the manifolds 23a and 23b, the exiting microchannel 26 to the waste well 27. By designing microchannel with different depths, widths and extending angles in the flowing path of the microfluid, the microfluid can be distributed as those shown in FIG. 7(a) to FIG. 7(d) when it is flowing from the upstream of the main microchannel to the downstream of the same. In FIG. 7(a) to 7(d), the blacked areas represent the areas that are filled by the microfluid, which can be clarified with reference to FIG. 5, FIG. 5A and FIG. 5B.

In FIG. 7(a), as soon as a microfluid is filled into the loading well 21, it will be affected by interactions between atmosphere pressure through the via hole 211, gravity, absorption from the polymer fiber in the main microchannel 22 and thus driven to flow automatically and continuously out of the loading well 21 and into the main microchannel 22. Since the width W3 of the manifold 23a is equal to the width W2 of the main microchannel 22 while its depth h3 may or may not be the same as the depth h2 the main microchannel 22, a portion of the microfluid will flow into the manifold 23a while the rest of the microfluid keep flowing in the main microchannel 22 and into the recess 25. When the flowing microfluid reaches the recess 25, the flowing microfluid is resisted and thus blocked by the recess 25 owing to its depth h5 and extending angle β5. However, as there is still microfluid keep flowing out of the loading well 21, the flowing microfluid will be diverted to flow into the manifold 23a where it is affected by the pulling force caused by the atmosphere pressure through the via hole 241, and eventually fill the whole manifold 23a, but the pulling is not large enough for driving the microfluid to flow into the via hole 241. After the manifold 23a is filled, the restriction area 24, formed at the tail of the manifold 23a can function as the recess 25 for causing a resisting force for preventing the microfluid from flowing into the restriction area 24 by its depth h4, diameter W4 and extending angle β4. Moreover, since the depth h4 and diameter W4 of the restriction area 24 is larger than the depth h5, diameter W5 of the recess 25, the resisting force caused by the restriction area 24 is larger than that caused by the recess 25. Therefore, instead of flowing into the restriction area 24, the flowing microfluid will overcome the resisting of the recess 25 and keep flowing downstream the main microchannel 22.

In FIG. 7(b), when the microfluid overcomes the resisting of the recess 25 and keeps flowing downstream the main microchannel 22, the successive manifolds 23a and 23b will be filled orderly in a manner similar to that described in FIG. 7(a). Eventually, when the resisting of the last recess 25 on the main microchannel 22 is overcome, the microfluid will flow into the exiting microchannel 26. As seen in FIG. 7(b), there is one more recess 25 being arranged on the exiting microchannel 26 at a position in front of the waste well 27, the microfluid flowing in the exiting microchannel 26 will be temporarily blocked from flowing into the waste well 27 by the recess's depth h5, and extending angle β4 until each and every manifold 23a, 23b is filled with microfluid so as to prevent the microfluid from being sucked dry by the absorption material 271 filled in the waste well 27.

In FIG. 7(c), when the resisting of the recess 25 in front of the waste well 27 is overcome, the excess microfluid is going to be absorbed rapidly by the absorption material 271. It is noted that the absorbing force caused by the absorption material 271 should be larger than that of the polymer fiber filled in the exiting microchannel 26; in addition, as there is no such material with water absorption ability being filled in the manifolds 23a and 23b, the microfluid filled in the manifolds 23a and 23b can be sucked out from the manifolds 23a, 23b by the absorption of the absorption material 271 and the polymer fiber, as shown in FIG. 7(c). Thereby, the microfluid filled in the parallel-extending manifolds 23a and 23b can be sucked to flow out of the manifolds 23a, 23b starting from those situated at the upstream of the main microchannel 22 to those situated at the downstream of the main microchannel 22 orderly, and flow into the main microchannel 22, the exiting microchannel 26 and finally into the waste well 27 where it is absorbed by the absorption material 271. After all the microfluid is absorbed by the absorption material 271 as shown in FIG. 7(d), the wetted absorption material 271 can be replaced and substituted by a new absorption material 271. However, it is noted that although there can be a variety of different microfluids to be used in the microfluidic apparatus of the invention for performing any biochemical analysis or the like, all those microfluids used in the microfluidic apparatus can be absorbed by a same absorption material 271; and after the biochemical analysis is completed and all the used microfluids are stored in the waste well, the microfluidic apparatus had accomplished what it is designed to do and thus can be disposed.

Hence, the accurate and autonomous quantification and separation of the microfluid as well as the performing of specific biochemical testing and analysis are achieved during the microfluid flowing in and out the plural manifolds 23a, 23b. The above embodiment is used only for illustration, other modifications can be achieved by configuring the microfluidic apparatus with main microchannel 22 as well as the manifolds 23a, 23b with different length, width and depth according to the reaction time requirements, the type of microfluid used, the type of biochemical testing and analysis to be performed, which are not to be regarded as a departure from the spirit and scope of the invention.

The characteristic of the embodiment shown in FIG. 5 is that: the design enables the microfluid to flow in and out the manifolds 23a and 23b in an automatic manner so that no addition microfluid collection process is required and thus no addition fluid collection devices/components are required to be configured inside the restriction areas 24. Consequently, the microfluidic apparatus not only is ease to operate, but also is simple in structure.

From the above embodiments, it is noted that not only the channel with low resistance in the aforesaid autonomous microfluidic apparatus is flooded by the microfluid flowing therein, but also the flowing microfluid will fill the whole microchannel structure formed in the apparatus. Moreover, accurate quantification and separation of the microfluid can be achieved using the plural manifolds since each manifold will be filled completely by the flowing microfluid and the dimension of each manifold, i.e. its length, width and depth, are specified designed for containing the microfluid of a specific amount. In addition, since the resisting of the recess 25 in front of the waste well 27 is used for ensuring each and every manifold 23a, 23b is filled completely by the flowing microfluid and after each manifold is filled, the excess microfluid remaining in the main microchannel 22 and the exiting microchannel 26 is going to be absorbed and drained to the waste well 27 by the cooperation of the absorption material 271 in the waste well 27 and the polymer fiber in the main microchannel 22. During the draining of the excess microfluid, as the cross section areas of the manifolds 23a, 23b are different from those of the main microchannel 22 and the exiting microchannel 26, the absorbing force caused by the absorption material 271 and the polymer fiber can only function to drain the excess microfluid remaining in the main microchannel 22 and the exiting microchannel 26 and is not going to affect the microfluid containing in the manifolds 23a, 23b, so that the goal of autonomous separation is achieved. Furthermore, the recesses 25 formed in the microfluidic apparatus are working as passive valves against the flowing in the main microchannel 22 and the exiting microchannel 26. In another word, the microfluidic apparatus of the invention can achieve autonomous separation without the help of any active parts, but only by specifically designing it microchannels with different cross section areas and by the interactions between gravity, adhesive force and surface tension.

In addition, according to the material of the microfluidic apparatus and the microfluid used, the surfaces of the main microchannel 22, the exiting microchannel 26 and the manifolds 23a, 23b are processed by a hydrophile/hydrophobic coating process for smoothing the flowing of the microfluid. It is noted that after the microfluid is quantified and separated in the microfluidic apparatus, the separated sections of microfluid are isolated from each other by independent valves and are not going to have any interference from each other so that each section can be used for an independent testing.

Since accurate quantification and separation of the microfluid can be achieved using the plural manifolds, its length, width and depth, are specified designed according to the type of microfluid used, and the amount of microfluid required for the biochemical testing and analysis to be performed. For clarity, the microchannel design used in the embodiment shown in FIG. 5, FIG. 5A and FIG. 5B is listed in the following table:

| | width | depth | length |
|---|---|---|---|
| Loading well 21 | 5.5 mm | 3.0 mm | 5.5 mm |
| Main and exiting microchannels | 1.0 mm | 1.0 mm | 48.0 mm |
| manifolds 23a | 1.0 mm | 0.5 mm | 18.0 mm |
| Restriction area 24 | 3.5 mm | 2.0 mm | 3.5 mm |
| Recess 25 | 1.0 mm | 0.3 mm | 1.0 mm |
| Waste well 27 | 6.0 mm | 5.0 mm | 6.0 mm |

Thus, by the microchannel design listed in the above table, the separation and quantification as those shown in FIG. 7(a) to FIG. 7(d) can be achieved.

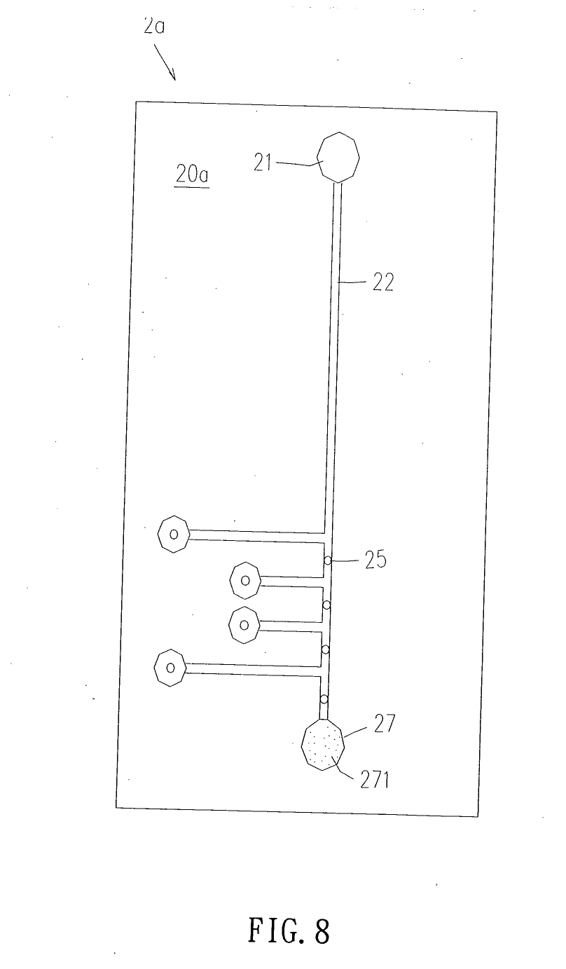

Please refer to FIG. 8, which is a front view of an autonomous microfluidic apparatus according to a second embodiment of the invention. Although the microfluid apparatus of FIG. 8 is designed basing on that shown in FIG. 5. there are still differences between the two.

First, the loading well 21 of the microfluidic apparatus 2a of FIG. 8 is not configured with the via hole as that is in the apparatus 2 of FIG. 5. It is known that the via hole is used for enabling the microfluid to flow out of the loading well 21 smoothly, however, when the inclination angle of the substrate 20a is large enough and the load well 21 of the microfluidic apparatus 2a is designed to channel with atmosphere, the affection from gravity upon the microfluid will be larger that the adhesive force of the microfluid upon the loading well 21 so that the microfluid will flow smoothly out of the loading well 21.

Secondly, the microfluidic apparatus 2a of FIG. 8 is not configured with the exiting microchannel 26 as that is in the FIG. 5. That is, the waste well 27 is connected directly to the tail of the main microchannel 22, which is simply a modification of the microfluidic apparatus of FIG. 5.

Moreover, there is no recess 25 being formed right at the connection of the main microchannel 22 and the waste well 27 as there is in FIG. 5. Although the recess 25 can prevent the microfluid from flowing into the waste well 27 too soon and too fast, however, as the cross section area of the main microchannel 22 is different from that of the waste well 27 and there is an absorption material stuffed in the waste well 27, so that when the affect of the weight of the microfluid matches the inclination angle of the substrate 20a, the recess 25 can be omitted.

Other than the aforesaid differences, other structures as well as their functionalities in the microfluidic apparatus of FIG. 8 are the same as those shown in FIG. 5, which can refer to those description relating to FIG. 5, FIG. 5A and FIG. 5B, and thus are not described further herein.

To sum up, the microfluidic apparatus of the invention uses the benefits of the physical scaling laws of the microworld and the interaction between gravity, adhesive force and surface tension for achieving autonomous separation and quantification, which has the following advantages: no active parts required; autonomous separation can be achieved simply by gravity, adhesive and its geometrical structure design; it can prevent microfluid containing in each manifold from interfering with each other; while filling two microfluids into the microfluidic apparatus one after another according to the specific order, it is able to prevent the two successive microfluids from mixing with each other; the manufacturing of the microfluidic apparatus is simple and has good flexibility that enables the microfluidic apparatus to be adapted for all kinds of microfluidic system easily; the volume of each separated section of microfluid can be defined with high accuracy; it can be used for performing experiences in batch process.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

What is claimed is:

1. An autonomous microfluidic apparatus, comprising:
a substrate; and
a microchannel structure, arranged on the substrate and further comprising:
a main microchannel;
a loading well, formed on the main microchannel;
a plurality of manifolds, each channeling with the main microchannel;
at least a passive valve, each being disposed at the main microchannel at a position between any two neighboring manifolds of the plural manifolds; and
a plurality of restriction areas, formed at ends of the plural manifolds in respective.

2. The autonomous microfluidic apparatus of claim 1, wherein the depth of the main microchannel is different from those of the plural manifolds.

3. The autonomous microfluidic apparatus of claim 1, wherein the lengths of the plural manifolds are not the same.

4. The autonomous microfluidic apparatus of claim 1, wherein the plural manifolds are arranged parallel with each other.

5. The autonomous microfluidic apparatus of claim 1, wherein the loading well is connected to at least a via hole, provided for enabling the loading well to be subjected to the atmospheric pressure and thus exerting a specific pressure to the microfluid in the loading well.

6. The autonomous microfluidic apparatus of claim 1, wherein the cross section area of each restriction area is different from that of the manifold where it is connected with.

7. The autonomous microfluidic apparatus of claim 1, wherein the passive valve is substantially a recess.

8. The autonomous microfluidic apparatus of claim 1, wherein the main microchannel is configured with a waste well, being an area situated at a downstream end of the main microchannel and filled with a material selected from the group consisting of a polymer fiber, materials with. water absorption ability, and the combination thereof.

9. The autonomous microfluidic apparatus of claim 8, wherein the cross section area of the waste well is different from that of the main microchannel where it is connected with.

10. The autonomous microfluidic apparatus of claim 8, wherein an exiting microchannel is arranged at a position between the main microchannel and the waste well in a manner that it is extending perpendicular to the main microchannel.

11. The autonomous microfluidic apparatus of claim 10, wherein the exiting microchannel is extending parallel to the plural manifolds.

12. The autonomous microfluidic apparatus of claim 10, wherein a passive is arranged at the exiting microchannel at a position proximate to the waste well.

13. The autonomous microfluidic apparatus of claim 12, wherein the cross section area of the passive valve is different from those of the exiting microchannel and the waste well.

14. The autonomous microfluidic apparatus of claim 12, wherein the passive valve is substantially a recess.

15. The autonomous microfluidic apparatus of claim 1, wherein the main microchannel is filled with a material selected from the group consisting of a polymer fiber, materials with water absorption ability, and the combination thereof.

16. The autonomous microfluidic apparatus of claim 1, wherein the substrate is a flat plate having the main microchannel to be formed thereon in equal depth.

17. The autonomous microfluidic apparatus of claim 16, further comprises: a slope structure, used for sloping the substrate and thus forming an included angle between the sloped substrate and a datum water level so as to slope the main microchannel from the downstream side thereof to the upstream side thereof with increasing height according to the included angle.

18. The autonomous microfluidic apparatus of claim 1, wherein each of the plural manifolds is extending about perpendicular to the main microchannel.
